(19) United States
(12) Patent Application Publication
(10) Pub. No.: US 2018/0344239 A1
(43) Pub. Date: Dec. 6, 2018

(54) Managing Evidence-Based Rules

(71) Applicant: Segterra, Inc., Cambridge, MA (US)

(72) Inventors: Gil Blander, Lexington, MA (US); Margaret Ploch, La Jolla, CA (US); Dexter Pratt, La Jolla, CA (US); Rony Sellam, Brookline, MA (US)

(21) Appl. No.: 15/774,864

(22) PCT Filed: Nov. 10, 2016
(86) PCT No.: PCT/US2016/061290

§ 371 Date: May 9, 2018

Related U.S. Application Data
(60) Provisional application No. 62/255,082, filed on Nov. 13, 2015.

(51) Int. Cl. A61B 5/00; A61B 5/11; G16H 50/20

(52) U.S. Cl. CPC A61B 5/486; G16H 50/20; A61B 5/1118; A61B 5/681

(57) ABSTRACT

Technologies described in this document can be embodied in a computer-implemented method that includes receiving, via an interface displayed on a computing device, an identifier associated with a publication, and retrieving, by one or more processing devices based on the identifier, a set of attributes associated with the publication. The method also includes receiving, via the interface, one or more rule parameters representing a rule based on the publication, and receiving a set of health-related parameters representing a physical condition. The method further includes determining, based on at least a portion of the health-related parameters and the one or more rule parameters, whether the rule is applicable to the physical condition, and presenting a representation of the rule on an output device as a part of a list of rules in response to determining that the rule applies to the physical condition.

### TECHNICAL FIELD

This document describes technologies related to evaluating health, wellness, and fitness parameters, and producing personalized recommendations.

### BACKGROUND

Various biomarkers and physiological markers from the human body along with parameters related to individual demographics and behavior can be used in evaluating health, wellness and fitness of individuals.

### SUMMARY

In one aspect, this document features a computer-implemented method that includes receiving, via an interface displayed on a computing device, an identifier associated with a publication, and retrieving, by one or more processing devices based on the identifier, a set of attributes associated with the publication. The method also includes receiving, via the interface, one or more rule parameters representing a rule based on the publication, and receiving a set of health-related parameters representing a physical condition. The method further includes determining, based on at least a portion of the health-related parameters and the one or more rule parameters, whether the rule is applicable to the physical condition, and presenting a representation of the rule on an output device as a part of a list of rules in response to determining that the rule applies to the physical condition. The method can also include determining, based on demographics, genetics, health-related parameters, behavior tracking, and past history, whether the rule applies to the individual.

In another aspect, this document features a system that includes a memory device and an analysis engine. The analysis engine includes one or more processing devices, and is configured to receive, via an interface displayed on a computing device, an identifier associated with a publication, and retrieve, based on the identifier, a set of attributes associated with the publication. The analysis engine is also configured to receive, via the interface, one or more rule parameters representing a rule based on the publication, and receive a set of health-related parameters representing a physical condition. The analysis engine is further configured to determine, based on at least a portion of the health-related parameters and the one or more rule parameters, whether the rule is applicable to the physical condition, and present a representation of the rule on an output device as a part of a list of rules in response to determining that the rule applies to the physical condition.

In another aspect, this document features one or more machine-readable storage devices storing instructions that are executable by one or more processing devices to perform various operations. The operations include receiving, via an interface displayed on a computing device, an identifier associated with a publication, and retrieving, based on the identifier, a set of attributes associated with the publication. The operations also include receiving, via the interface, one or more rule parameters representing a rule based on the publication, and receiving a set of health-related parameters representing a physical condition. The operations further include determining, based on at least a portion of the health-related parameters and the one or more rule parameters, whether the rule is applicable to the physical condition, and presenting a representation of the rule on an output device as a part of a list of rules in response to determining that the rule applies to the physical condition. The operations can further include determining, based on check-in data from the user, how successful the user has been in following the rule, and determining whether the user should receive further rules to promote success.

Implementations of the above aspects of the technology can include one or more of the following features.

The list of rules can include one or more additional rules also applicable to the physical condition. A rank of the rule within the list can be determined. The rank can be determined based on at least a portion of the set of attributes associated with the publication, or based on a degree of applicability of the rule to the physical condition or based on a degree of applicability of the rule to the individual. One or more controls for initiating edits to the one or more rule parameters can be presented via an interface. The one or more rule parameters can include at least one of: a set of one or more biomarkers or genetic markers associated with the rule, a set of one or more physiological markers associated with the rule, a set of demographic and behavior data, a set of one or more intermediate parameters associated with the rule and a set of data about user compliance with the rule. The one or more rule parameters can also include at least one condition defined based on the set of one or more biomarkers, the set of one or more physiological markers, and/or the set of one or more intermediate parameters. The condition can be used to determine whether the rule is applicable to the physical condition. The one or more rule parameters can include information on an output associated with the rule. The one or more additional rules can be retrieved from a rules database stored on one or more storage devices, and a determination can be made, based on one or more of the health-related parameters, that the one or more additional rules are also applicable to the physical condition. A recommendation for improving the physical condition can be generated based on the rule. A representation of the rule can be stored in a database. The identifier can include at least one of: i) a PubMed identifier associated with the publication, ii) a hyperlink to a storage location, or iii) a filename associated with an attachment that includes the publication. The set of attributes can include one or more of: a title of the publication, an identification of a journal associated with the publication, a date associated with the publication, an author of the publication, and an institution associated with the publication. The set of attributes can include an impact factor of a journal associated with the publication. Information on one or more user characteristics for a user associated with the physical condition can be received, and a determination can be made, based at least on a portion of the information on the one or more user characteristics, whether the rule is applicable for the user. The information on one or more user characteristics can include at least one of: demographic information, user goals, and information on user behavior. Retrieving the set of attributes can include accessing an electronic repository storing information about the publication, and retrieving at least a portion of the set of attributes from the electronic repository responsive to identifying the publication using the identifier. Retrieving the set of attributes can include accessing an electronic copy of the publication, and retrieving at least a portion of the set of attributes from the electronic copy of the publication using an optical character recognition (OCR) process. The set of health-related parameters can be received via a user interface displayed on a computing device. The set of health parameters can be based on measurements obtained using a wearable device.

In some implementations, the technologies described herein may provide one or more of the following advantages.

Personalized recommendations and information for a user (e.g., recommendations and information based on one or more of demographics, biological markers, physiological markers, health and lifestyle-related parameters and goals) can be updated based on new results and studies, but only upon validating that such results are applicable to the user. Reliability of the new results and studies can be automatically estimated based on analyzing identifiers associated with the publication sources of the results and studies. Applicability of various rules to particular users or user groups, as well as redundancies and inconsistencies associated with the rules, can be analyzed prior to validating new rules. The interface described in this document can be used for creating, testing, validating, editing, deleting, or otherwise managing rules without manual reprogramming, which may be time consuming, expensive, or error-prone.

Personalized recommendations and advice for a user can be updated based on compliance data from the user, either based on a direct user check-in process, or based on data automatically received from various sources including fitness devices such as a wireless scale or activity tracker. In some cases, the system can be configured to provide responsive feedback to the user based on evaluating a set of rules. For example, a user who receives a recommendation to eat wheat germ seven days a week, but fails to do so, might receive an updated recommendation. In this case, if the user is known to consume fruit and/or yogurt (and not being allergic to them), the updated recommendations may suggest sprinkling wheat germ on the fruit or yogurt consumed by the user.

In some cases, a user may receive advice/recommendations through an interactive chat. The content of the chat can be determined, for example, by one or more of: demographics, biological markers, genetic markers, physiological markers, health and lifestyle-related parameters and goals, user behavior, and the user's history of results and behavior. For example, if a user has failed to take a recommended supplement regularly, the interactive chat may suggest storing some of the supplement at the user's workplace, so that they can take it at work if they forget to take it at home.

The trends shown in past results and behavior may predict future outcomes. For example, the system can be configured to calculate two average points based on a predetermined number of previous results (e.g., three, four, five, or more previous results) of the user for a biomarker. The slope between these points can be projected into the future, for example, to predict the likely direction and rate of change of future results. The trend of future results may be determined by one or more of past biomarker results, past behavior, user characteristics, genetic markers, and physiological parameters. Recommendations for the user may be determined based on the predicted future biomarker results.

### BRIEF DESCRIPTION OF THE DRAWINGS

FIGS. 1A and 1B show examples of block diagrams of a system for managing a rules database.

FIGS. 2A and 2B are examples of user interfaces associated with retrieving and presenting sources that form the basis of the rules in the rules database.

FIGS. 3A-3C are examples of user interfaces for managing rules

FIG. 4 is an example of a user interface for managing one or more parameters associated with a rule.

FIG. 5 is an example of a user interface for presenting an output of a simulation process in accordance with technology described herein.

FIGS. 6A-6D are examples of user interfaces for presenting recommendations and information to a user.

FIG. 7 is an example of a user interface for presenting a dashboard associated with the rules.

FIG. 8 is a flowchart representing an example process for managing the rules.

FIG. 9 is a block diagram of an example of a computing system.

### DETAILED DESCRIPTION

With the advent of renewed focus on personal health and wellness, widespread research on nutrition, exercise, lifestyle, supplements, etc. has produced, and continues to produce, numerous studies, research papers, and articles that present health and wellness-related advice. Many of these publications are publicly available to individuals seeking to improve their health and performance. However, the results presented in these publications are based on individuals or groups of individuals with specific sets of health-attributes, and it is challenging to evaluate whether a particular advice or study is applicable to a particular individual. In some implementations, evaluating the results or advice from a given study for a particular individual may require not only the details of specific health-attributes used in the study, but also specific information on the health status of the particular individual. For example, one particular study may generally advocate consuming one half of a fresh avocado for lunch to reduce food craving. However, for people with latex allergies, consuming avocados may have adverse side effects. Therefore, to evaluate the applicability of the particular study for an individual, allergy information pertaining to the individual may be needed.

In some implementations, a rules database can be used to generate personalized advice on nutrition, exercise, lifestyle, supplement and other health-related issues. Rules from the database can be personalized for individuals (or group of individuals), based on specific inputs on one or more parameters associated with the individual (or group of individuals). Examples of such parameters include biomarkers, such as markers in blood test data and genetic information, physiological markers, demographics, health and lifestyle parameters, and personal goals of the individuals. In one illustrative example one parameter (e.g., a biomarker like vitamin D) may affect one or more of other parameters. For example, low vitamin D may affect testosterone levels in men and bone health in both sexes. In another example, vitamin D production in the skin may vary with ethnicity such that individuals of one particular ethnicity (e.g., African-Americans) may need to consume more vitamin D in supplement form to reach a predetermined optimal level. This document describes technology for managing and augmenting a rules database based on new studies and results as they become available. The technology facilitates creating and editing a library of sources that may be used to support present or future rules. The technology also enables users to create source entries that include a variety of data that may be used to sort and rank the rules, for example, based on the corresponding publication sources.

Various relationships between the rules and the corresponding sources can be used within the rules database, including, for example, one-to-one, one-to-many, many-to-one, and many-to-many. For example, a single source may be used to support one or more rules, and/or one or more sources may be used to support a single rule. For example, a single publication may serve as the basis for a rule that suggests taking 3-5 mg melatonin to facilitate sleep in a new time zone, as well as for another rule that suggests taking meals 1-2 hours earlier than usual when traveling eastwards or 1-2 hours later than usual when traveling westwards. On the other hand, a rule that suggests using earplugs or other means of blocking out noise in order to improve sleep quality may be supported by two separate sources (e.g., a research article as well as the USOC Jet Lag Countermeasures and Travel Strategies Brochure).

In some implementations, management of the rules database is facilitated by providing an interface that allows for controlling an expert system without having to reprogram the system. This allows for health experts such as biologists, exercise physiologists, nutritionists, and other scientists to create new rules, simulate specific conditions (e.g., as represented by a set of parameters of an individual) to test validity and/or applicability of the rules, and refine the rules, for example, to tailor the rules for the specific conditions. In some implementations, the technology described herein includes a version control system that checks for inconsistencies and redundancies, and replaces outdated rules with more recent versions.

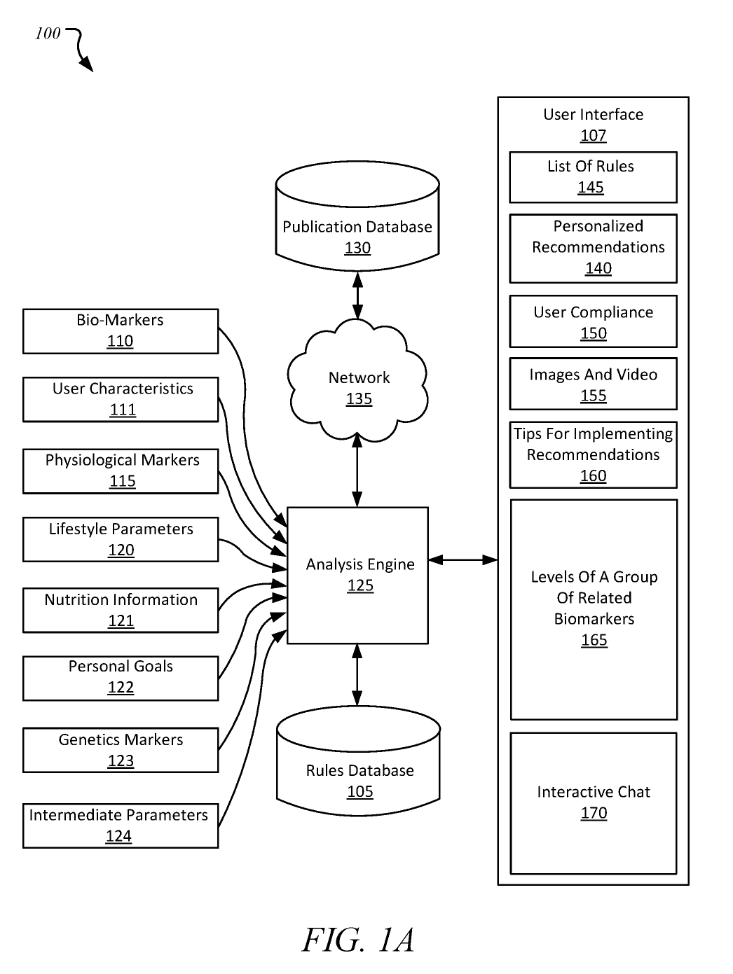

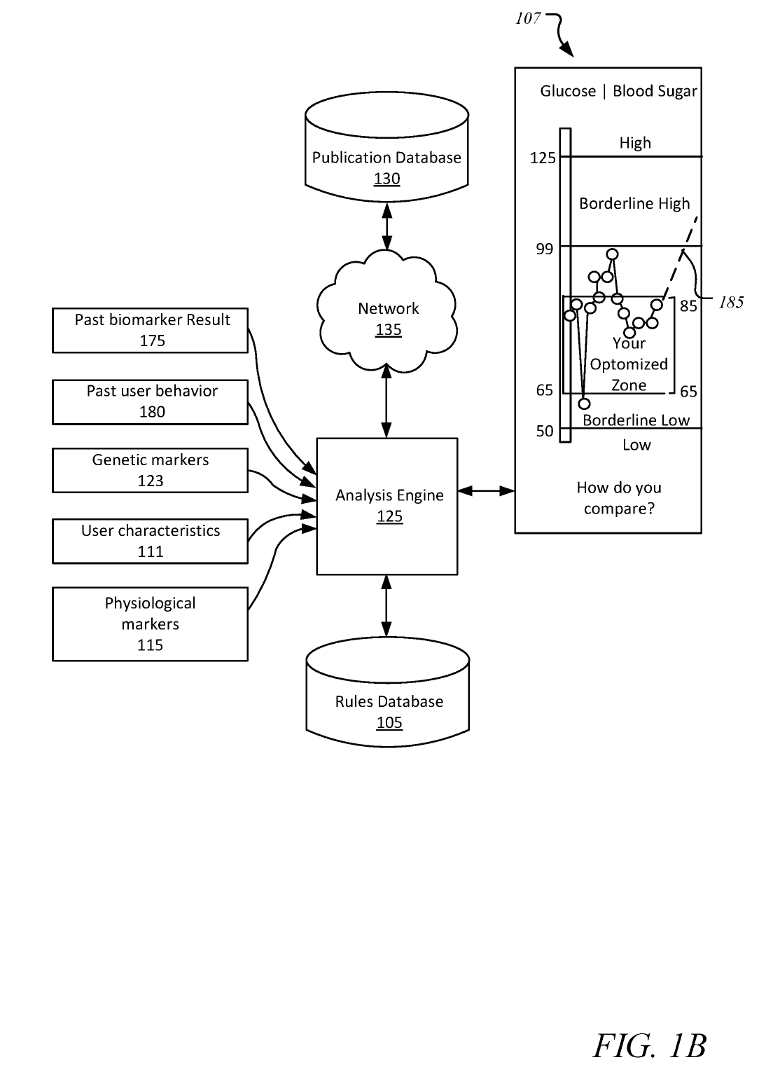

FIG. 1 shows a block diagram of an example system 100 for managing a rules database 105. The system 100 includes an analysis engine 125 in communication with the database 105. In some implementations, the analysis engine 125 can be configured to create one or more rules for storage within the rules database 105 based on information received via a user interface 107. The analysis engine 125 can also be configured to provide personalized recommendations to a user based on various input parameters. Examples of such input parameters can include one or more biomarkers 110, genetic markers 123, physiological markers 115, lifestyle parameters 120, nutrition information 121, and personal goals 122 of an individual. In some implementations, the various input parameters include one or more intermediate parameters 124 that may be used to create rules with complex interdependencies. Some of the intermediate parameters may not be visible to an end user, but defined or created by the analysis engine 125 in the process of creating, augmenting, modifying or otherwise editing one or more rules. In some implementations, the input parameters are based on measurements obtained from a human user. In some implementations, the input parameters can be provided as a representation of a hypothetical individual used in testing, validating, editing, or creating a rule.

The various input parameters can be obtained from various sources. For example, information about the various biomarkers 110, user characteristics 111, physiological markers 115, lifestyle parameters 120, and/or personal goals 122 can be obtained using one or more devices, such as wearable devices (e.g., smart watches or activity trackers). Such wearable devices can be configured to measure, compute, or otherwise provide information on various health/fitness related parameters such as heart rate, calorie consumption, calorie expenditure, distance walked, steps taken, electro cardiogram (ECG), or quality of sleep. The devices can also include non-wearable devices such as weight scales or other scales configured to provide information on weight, body-mass index (BMI), or water content of the body. In some implementations, the various input parameters may also be provided to the analysis engine 125 using, for example, personal computer devices or mobile computing devices configured to present an interface for a user to enter the various input parameters. In some implementations, the interface can include the user interface 107 shown in FIG. 1. In some implementations, the interface can be provided using one or more applications for measuring at least a portion of the various input parameters. In some implementations, the various input parameters can be based on user-input on one or more tests such as blood tests, urine test, sputum test, stool test, or other tests for determining levels of one or more biomarkers 110, genetic markers 123, physiological markers 115, and lifestyle parameters 120. In some implementations, at least a portion of the various input parameters can be obtained from a remote data source. For example, information on these markers and parameters can be obtained, for example, from the medical records of the individual based on appropriate permissions from the individual and from a remote storage location (e.g., a cloud storage system) storing such records.

In some implementations, the analysis engine 125 can be configured to access data for a plurality of input parameters that includes one or more goals of an individual, and identify a set of one or more out-of-range parameters or unmet goals. The analysis engine 125 can be further configured to identify one or more rules from the rules database 105, such that the identified rules are related to improving the corresponding levels of the one or more out-of-range parameters. The analysis engine 125 can also be configured to present, for example, on a display device, one or more personalized recommendations 140. The personalized recommendations can include, for example, one or more recommendations related to exercises, lifestyle, diet, supplement, and educational materials. In some implementations, the recommendations may be provided on a unified user interface 107 presented on a display device. The user interface 107 can be used to present various other information together with the personalized recommendations 140, including, for example, a list of rules 145, information on a level of user compliance 150, one or more images and videos 155 associated with the recommendations, information on tips for implementing the recommendations 160, and an interactive chat functionality 170. The chat functionality 170 may allow a user to interact with an online persona (e.g., an automated system configured to respond to user queries) that provides education and support to the user. In some implementations, the user interface 107 can also include a representation of levels of a group of related biomarkers, wherein the group of biomarkers are selected based on, for example, demographic, physiological, and/or other characteristics of the user.

In some implementations, more or less input parameters may be used by the analysis engine to present particular outputs. FIG. 1B shows one particular example implementation where the analysis engine 125 is configured to generate averages of most recent sets of results for a biomarker to calculate a predicted future trend. The analysis engine 125 can be configured to calculate such averages based on input parameters such as past biomarker results 175. Such averages can be combined with other input parameters such as one or more of past user behavior 180, genetic markers 123, physiological markers 115, and user characteristics 111 to determine a future trend 185 and a likely rate of change for the levels of the corresponding biomarker. This is illustrated in FIG. 1B with the specific example of changes in the level of glucose.

Examples of biomarkers 110 can include glucose, total cholesterol, high density lipoprotein (HDL), low density lipoprotein (LDL), triglycerides, testosterone, free testosterone, estradiol, dehydroepiandrosterone-sulfate (DHEA-S), prolactin, vitamin D, hemoglobin, calcium, parathyroid hormone (PTH), insulin-like growth factor such as IGF-1, tumor necrosis factor (TNF) such as TNF-alpha, pro-inflammatory cytokine such as IL-6, C-reactive protein (CRP), high sensitivity CRP (hsCRP), folic acid, vitamin B12, alanine aminotransferase (ALT), aspartate aminotransferase (AST), gamma-glutamyl transpeptidase, blood urea nitrogen (BUN), ferritin, sodium, zinc, white blood cells, potassium, creatine kinase (CK), sex hormone binding globulin (SHBG), cortisol, albumin, total iron binding capacity (TIBC), unsaturated iron binding capacity (UIBC), progesterone, luteinizing hormone, follicle-stimulating hormone, chromium, thyroid-stimulating hormone (TSH), magnesium, and markers that are part of the Complete Blood Count test, including hematocrit, mean cell hemoglobin, mean cell hemoglobin concentration, mean cell volume, red blood cell count, red blood cell distribution width, platelets, mean platelet volume, neutrophils (absolute), lymphocytes (absolute), monocytes (absolute), eosinophils (absolute), basophils (absolute), neutrophils (percent), lymphocytes (percent), monocytes (percent), eosinophils (percent), basophils (percent). In some implementations, a genetic marker 123 or telomere length can be used as a biomarker 110. Examples of genetic markers include single nucleotide polymorphisms (SNPs) in which a single allele is changed to a variant allele, or in which an allele is added or deleted. For example, a variant on one gene may be associated with greater chance for abdominal weight gain for an individual. The system may also look at related groups of SNPs and evaluate them. In some implementations, SNPs can be selected based on a likelihood of their affecting the level of the biomarkers being measured. The selected SNPs can include, for example, SNPs associated with food sensitivity, SNPs that may influence athletic activity and performance, SNPs that may influence particular goals, such as weight loss, better sleep, and/or injury prevention, and SNPs that are associated with overall wellbeing of individuals. Examples of SNPs that may be used in accordance with the above criteria are provided in Appendix A. A larger set of SNPs that may be used are provided in Appendix B. Examples of physiological markers 115 can include, for example, heart rate variability, pulse pressure, heart rate, BMI, blood pressure, weight, body fat percentage, and height. Examples of lifestyle parameters 120 can include activity level (e.g., amount of exercise done per day), smoker status (e.g., whether or not a smoker, time since quitting, number of cigarettes or other units smoked per day etc.). Examples of user characteristics can include demographic parameters (e.g., age, gender, and ethnicity) and user behavior. Examples of user behavior can include one or more of: number of caffeinated beverages consumed daily, frequency of consuming certain types of food such as dairy and red meat, type and amount of dietary supplements taken, and time spent in the sun daily. The analysis engine can also be configured to take into account changes in inputs, e.g., biomarkers, over time. In addition, the analysis engine can be configured to determine the averages of a number of the most recent results for a user, and use that trend to predict the user's future results.

Examples of intermediate parameters 124 can include parameters that are functions of one or more of biomarker measurements, health-related parameters, demographic data, diet, exercise, supplement and lifestyle parameters, activity level and/or user data. Examples of personal goals 122 can include user-defined objectives such as to sleep better, improve immune function, lose fat, gain muscle, maintain weight, build strength and power, build endurance, prevent injury/speed recovery, reduce stress, fight aging, sleep better, strengthen immune system, improve cognition, boost energy, improve digestion, improve sex life, improve bone health and/or increase endurance.

In some implementations, the analysis engine 125 can be configured to augment the rules database 105 by facilitating creation of one or more new rules based on new results/studies that may become available. Evidence of scientific support for the new results/studies can include, for example, publications such as peer-reviewed research articles, technical papers or datasheets, publications from government agencies such as the National Institutes of Health, and publications from regulatory bodies such as the US Olympic Committee. Evidence of scientific support may also include original research conducted by scientists using anonymized data from customers and from public data sources. In some implementations, the analysis engine 125 can be configured to retrieve one or more attributes of a particular publication based, for example, on an identifier associated with the particular publication. For example, the analysis engine can be configured to receive, e.g., via a user interface, an identifier associated with a publication, and retrieve the one or more attributes from a corresponding publication database 130. The publication database 130 can be identified, for example, based on the identifier associated with the publication. In some implementations, the publication database 130 is stored on a remote storage device, and connected to the analysis engine 125 over a network 135 (e.g., the Internet). In some implementations, the publication database can be stored on a local storage device (e.g., a storage device that also stores the rules database 105).

In some implementations, the analysis engine 125 can be configured to implement a review process that can be used to ensure, for example, that the rules are unambiguous, accurate, and supported by research evidence. Using such a process, various personnel including scientists are able to review a rule (possibly via multiple iterations) before the rule is finalized. For example, the analysis engine can present an interface that allows a scientist to define a rule and save a draft version of the rule on a storage device. The analysis engine 125 can also be configured to present a reviewable version of the rule, for example, to a personnel with approval capacity. This can be done, for example, upon verifying by the analysis engine 125, that the personnel indeed has approval capacity. In some implementations, the analysis engine 125 may have access to a database of authorized users and their respective levels of permissions. The analysis engine 125 may use information from such a database to determine if a personnel has permissions to review, approve and/or lock a given rule. In some implementations, the permissions can be specific to a rule or type of rule. In some implementations, one or more administrative users can have permissions to add new users, set and change permission levels, and delete users.

The approver may evaluate various components of the rule (including, for example, the publication source associated with the rule) via the interface presented by the analysis engine. Upon receiving an indication of approval, the analysis engine can be configured to change a status of the rule accordingly. Alternatively, upon receiving notification that one or more edits are to be made, the analysis engine 125 can be configured to change the status of the rule to indicate a draft status, and present the rule in an editable interface for a personnel (e.g., the original author) to make any edits. Approved rules can be presented by the analysis engine for a final review and “locked” to prevent any inadvertent changes. In some implementations, the analysis engine 125 may only use locked rules to generate recommendations for an end-user. If a locked rule needs to be changed for any reason, the analysis engine can present an administrator interface for unlocking the rule for editing, and updating the status of the rule to a draft status.

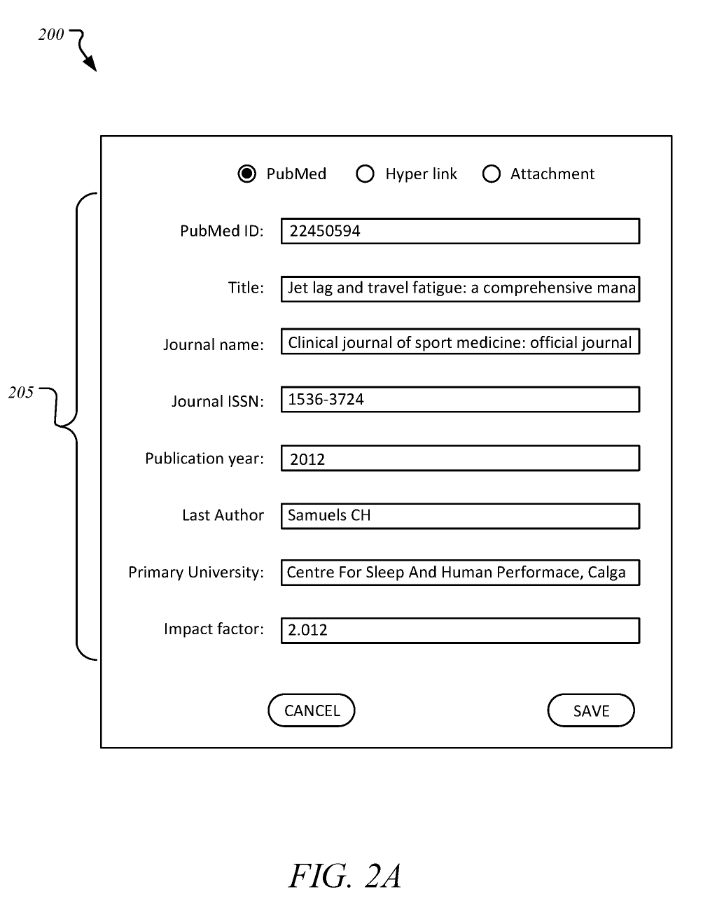

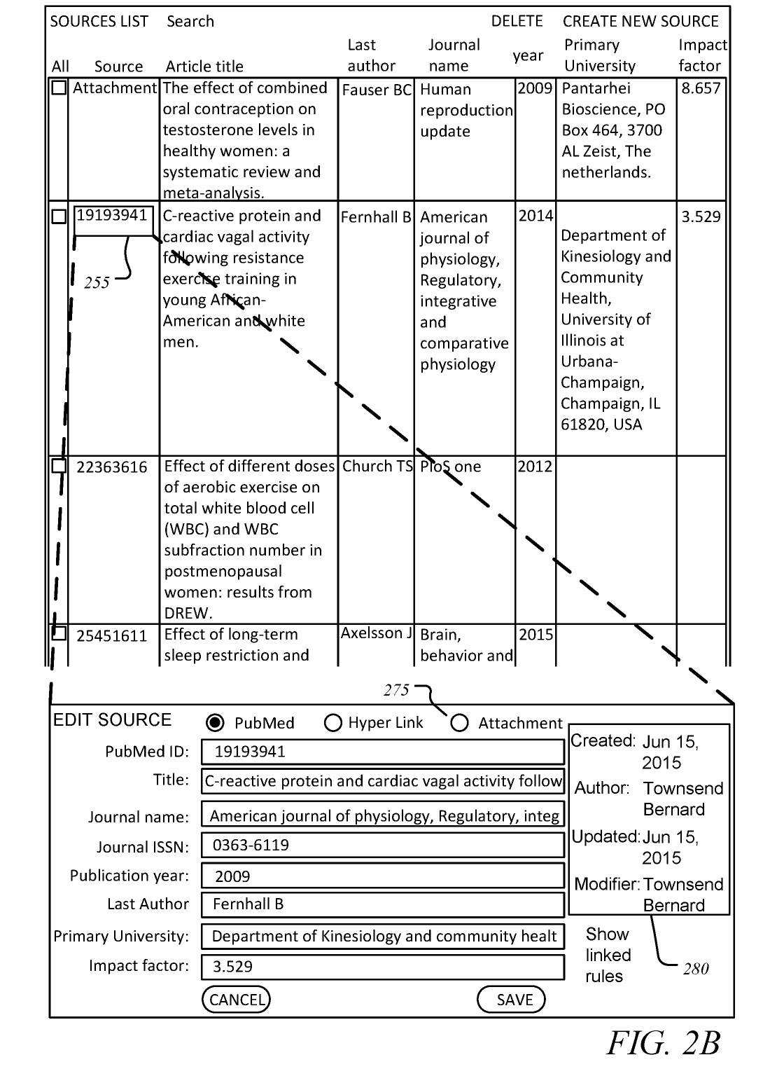

FIG. 2A shows an example of a user interface associated with retrieving one or more attributes 205 of a source or publication that forms the basis of a new rule. The identifier can include, for example, a unique identifier (such as a PubMed ID or uniform resource locator (URL)) associated with the source. In some implementations, the analysis engine 125 can be configured to automatically retrieve, based on the unique identifier, at least a portion of the one or more attributes 205. Examples of the attributes 205 can include article title, journal name, journal ISSN, name of author(s), affiliation of the author(s), date of publication, and impact factor of the corresponding publication source. In some implementations, the identifier can include an attachment (e.g., a copy of the publication) from which the analysis engine extracts at least a portion of the attributes 205 (e.g., using optical character recognition (OCR)).

FIG. 2B shows an example of a user interface 250 for presenting a library view of various sources accessible to the analysis engine 125. In some implementations, the interface 250 can include one or more controls for allowing various operations including, for example, searching and/or sorting the various sources based on parameters such as: type of source, title, author, publication name, publication date, author affiliation, and impact factor. The interface 250 can also include one or more controls for selecting a particular source for editing, deleting one or more sources, or creating a new source. In some implementations, activating a control 255 for selecting a particular source can launch a different interface (e.g., the interface 275) that presents the details of the corresponding source in editable form. In some implementations, the interface 275 can be configured to present a history 280 associated with the corresponding source.

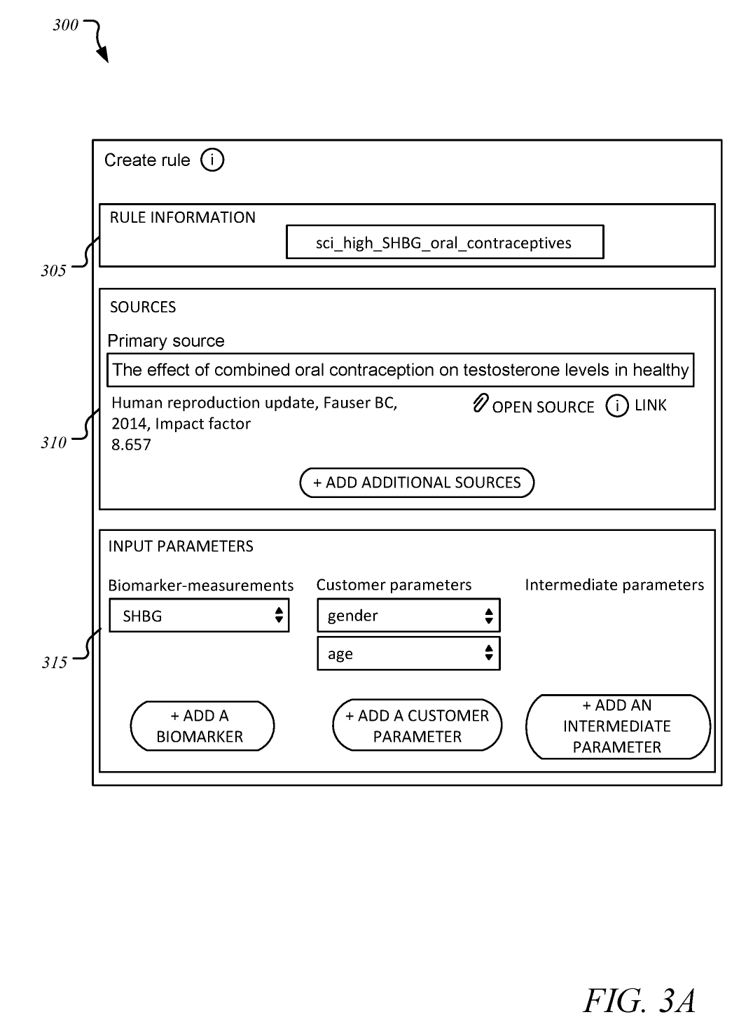

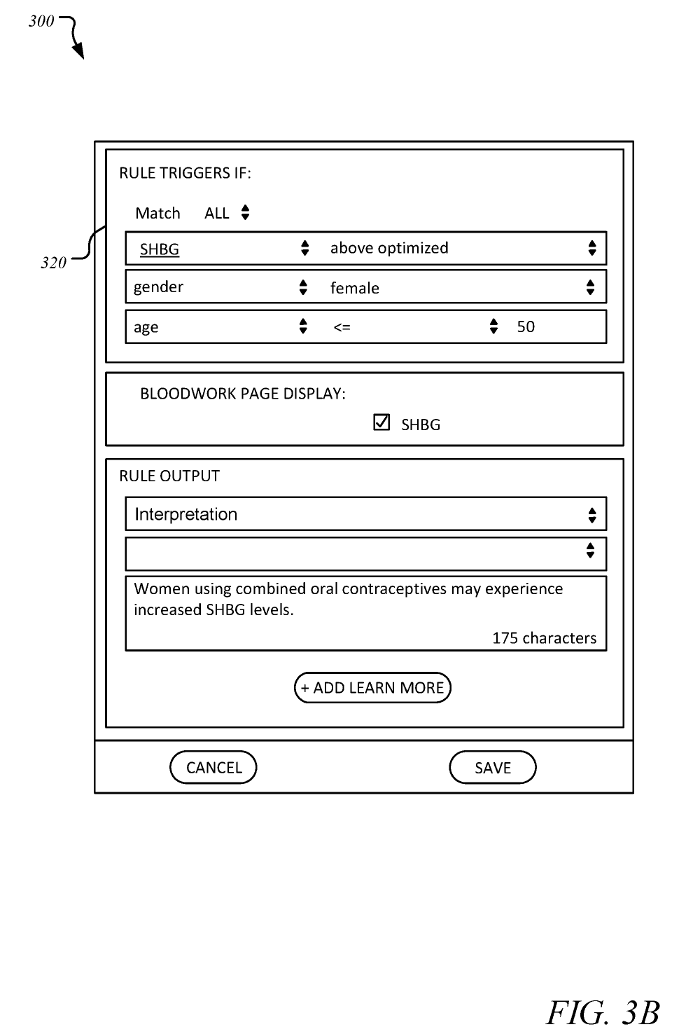

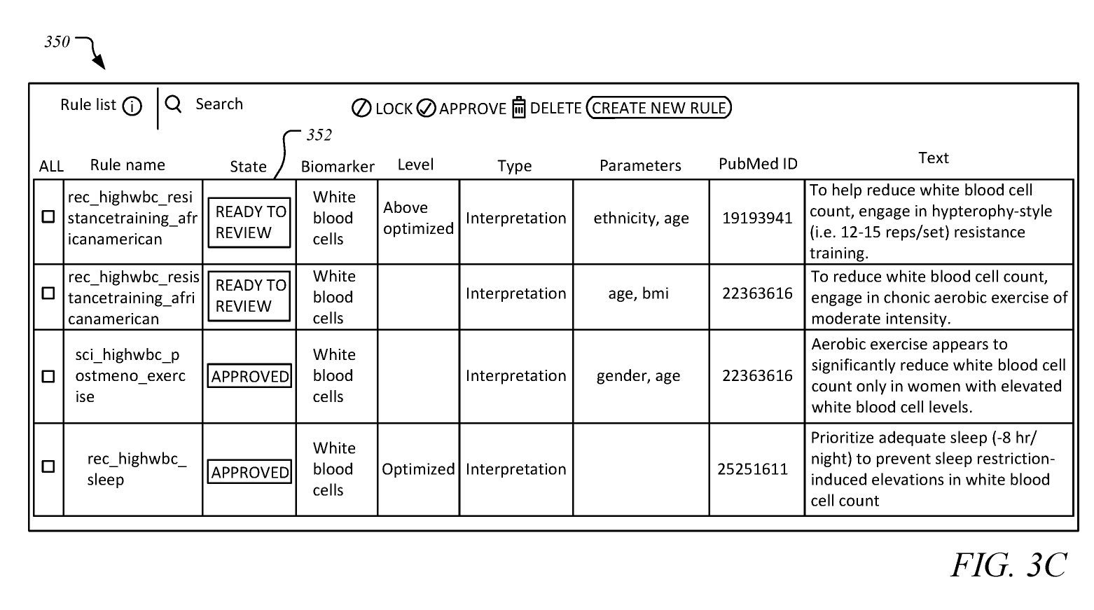

In some implementations, the analysis engine 125 can be configured to provide a user interface that allows creation of a new rule. Examples of such an interface 300 is shown in FIGS. 3A and 3B. The interface 300 can present one or more fields that allow a user to specify several attributes associated with the rule. The attributes include, for example, identification information 305 (e.g., a name for the rule), identification of one or more sources supporting the rule 310, and identification of one or more input parameters 315 associated with the rule. A rule may have one or more supporting sources, the details of which may be retrieved from the rules database 105. The particular example in FIG. 3A shows creation of a rule “sci_high_SHBG_oral_contraceptives” for providing recommendations to female users who have high SHBG levels, and take oral contraceptives. The recommendations are configured to inform such users that women taking oral contraceptives tend to have higher SHBG levels than women using non-hormonal contraceptive methods.

As shown in FIG. 3B, the interface 300 can be configured to present one or more fields that allow for entering one or more conditions 320 for triggering a rule. The interface 300 can also be configured to present one or fields that allow for specifying the output associated with the rule. For example, such specification can include the place(s) where the rule output will be displayed, a type of output, and content of the output. When saved, the rule can be added to the rules database 105, possibly in a draft form that may require later approval. In the particular example of FIG. 3B, the rule can be triggered if a user has a level of sex hormone binding globulin (SHBG) above a predefined “optimal” zone, and is a female of age less than 50 (and therefore likely to be using oral contraceptives). The output of the rule will display the recommendations related to SHBG with the users' bloodwork results. The type of output would be an interpretation or statement intended to educate the customer. In some implementations, additional information can also be provided, for example, by specifying a hyperlink to more information on SHBG. The rule output may also include a group calculation in which the level of various related biomarkers and in some cases user demographics, physiological markers, genetics and other user characteristics determine the evaluation of the group of biomarkers. The rule output may also include an interactive chat related to recommendations and user behavior.

In some implementations, the analysis engine 125 can be configured to generate the programming code for the rule and save the code together with the rule attributes in the database 105. In some implementations, generating the programming codes automatically results in an efficient process that reduces manual intervention, allows for automatic checks for inconsistencies and redundancies, and facilitates review of a library of rules to compare rules by output, constraints, and a variety of other parameters. In some implementations, the rules can be tested individually, as well as in various groupings.

Once one or more rules are created, the analysis engine 125 can be configured to store the rules in the rules database 105 and run the rules on data for various users to determine whether the rule is applicable for a particular end user for a particular result and/or goal. In some implementations, each rule can specify the type of output and the content of the output. For example, the output of a rule may be one of the following: an actionable recommendation that an end user may follow to help improve a biomarker level or a physiological marker level, or to advance toward a personal goal. The output of a rule can also include one or more of the following: (i) a statement intended to educate the end user about a biomarker, physiological marker, or goal, (ii) an explanatory paragraph about the biomarker, physiological marker or the goal and the end user's status, (iii) a link to another document, such as a blog post, to provide the end user with additional information, (iv) notifications, specific to a particular time or location to remind the end user of relevant actions or goals as determined by analysis engine 125, or (vi) other outputs related to the biomarker, physiological, lifestyle and goal information related to each individual. Other types of information may also be provided to the end user, including, for example, specific notifications, menu planning, exercise advice and lifestyle recommendations.

The rules in the rules 105 can be presented, for example, via an interface. This is depicted in FIG. 3C, where the interface 350 presents a library view of at least a subset of rules stored in the rules database 105. The interface 350 can include, for example, a status notification 352 for each of the presented rules, the status notification representing whether the corresponding rule is being reviewed, has been approved, is in draft form, or has been locked to prevent unauthorized changes. The library view in interface 350 can allow a user (e.g., a system administrator) to search and/or sort the rules on a variety of attributes including, for example, rule name, status notification, associated biomarkers, associated levels, output type, parameters, source, and output content. Users with appropriate permissions may use the library view to approve, lock, or delete one or more rules.

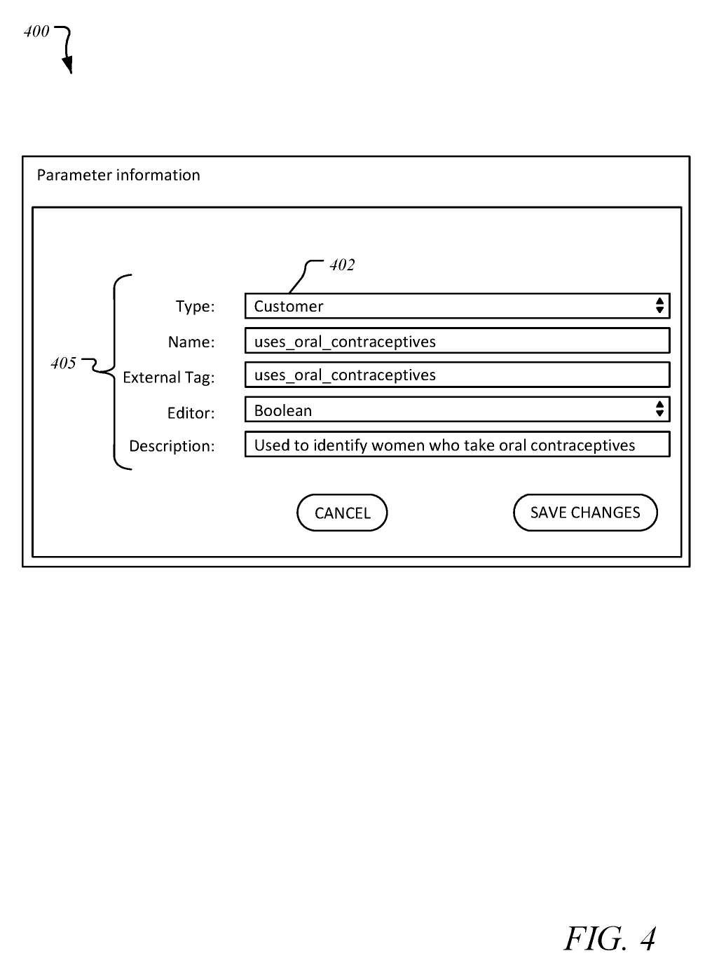

FIG. 4 is an example of a user interface 400 for managing parameters associated with one or more rules. The interface 400 allows a user (e.g., a scientist) to provide specifications about one or more parameters used in defining or modifying one or more rules. In this example, the parameters relate to customer data, as may be specified by the field 402. Examples of fields 405 in the interface 400 can include, for example, a name of the parameter, a tag associated with the parameter (e.g., a reference used for the parameter in creating or editing a rule), a type of value for the parameter (e.g., numerical or Boolean), and a description of the parameter. In some implementations, the interface 400 (or another interface) can be configured to allow users to define one or more intermediate parameters based on, for example, one or more of biomarker measurements, goals, demographics, and health- and lifestyle-related parameters. The specified parameters can be stored in a database (e.g., the rules database 105) and presented to a user, for example, as a library of parameters. The user (e.g., a scientist) can use such a library view of parameters, for example, to search and sort parameters in creating and/or modifying one or more rules.

In some implementations, the analysis engine 125 can be configured to implement a simulator that allows personnel (e.g., scientists) to run one or more tests on the rules. For example, the analysis engine can be used to test whether one or more rules are correctly triggered under applicable conditions, and/or whether the rules produce the expected output when triggered. In one example, a scientist may select a one or more biomarker(s), corresponding measurements, and/or one or more input parameters to review the set of rules that are triggered by the input data. Referring to FIG. 1, such a list of rules 145 may be presented using the user interface 107. In some implementations, the analysis engine can be configured to display one or more rules and outputs that would apply to a specific individual whose biomarker data, physiological data, lifestyle and other parameters are provided as input parameters. In some implementations, the analysis engine can be configured to allow the display of one or more rules related to a specific biomarker or individual goal. The analysis engine can be configured to display rules related to, for example, a combination of a specific demographic, food, exercise, lifestyle, supplement or other input parameters.

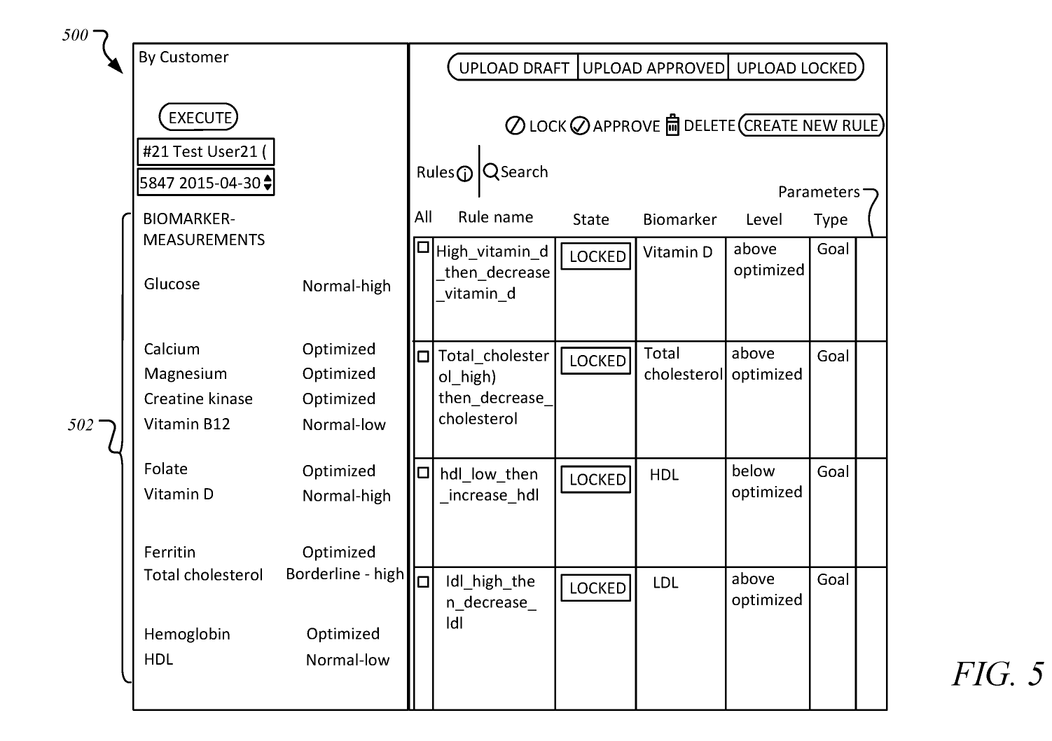

FIG. 5 shows an example of an interface 500 for presenting an output of a simulator. The interface 500 includes, for example, one or more controls that allow a personnel (e.g., a scientist) to choose a representative user (based, on for example, the levels of various input parameters 502) for the analysis engine to present one or more rules that would apply to the representative user's data. In some implementations, the representative user can be selected from user profiles stored in a database. In some implementations, the interface 500 can be configured to receive one or more rules, (the status for which may be draft, approved, or locked), and one or more input parameters associated with the rules. The input parameters can include, for example, biomarker and/or physiological measurements, lifestyle parameters etc., for a real or hypothetical individual. The interface 500 can also be configured to present an output of the simulator. For example, the interface 500 can be configured to present one or more rules that apply to the set of input parameters 502.

In some implementations, the rules presented on the interface 500 can be based on a ranking. In some implementations, the analysis engine 125 can be configured to compute the ranking based on one or more of: (i) the impact factor of the journal in which the primary study was published, (ii) number of times the study was cited, (iii) demographic, biomarker, physiological, exercise and lifestyle parameters, dietary restrictions, and health-related goals of the user, (iv) date of the primary study supporting the recommendation, (v) number of supporting studies, (vi) kind of study (double blind, prospective, meta-analysis, review, etc.), (vii) number of subjects in that experiment, and/or (viii) Intervention effect. In some implementations, the recommendations provided based on the rules can also be ranked based on the rank of the corresponding rules. In some implementations, the ranking can be based on a personalization grade of the rule, a higher value of which implies that the more specific the rule is to the user. For example, a rule for a runner who is a vegetarian is less personalized than a rule for a runner who is a female vegetarian between the ages of 18-50. In some implementations, the rules presented on the interface 500 can be based on learning from a user's choices and success and then applying that learning to derive rules for other users.

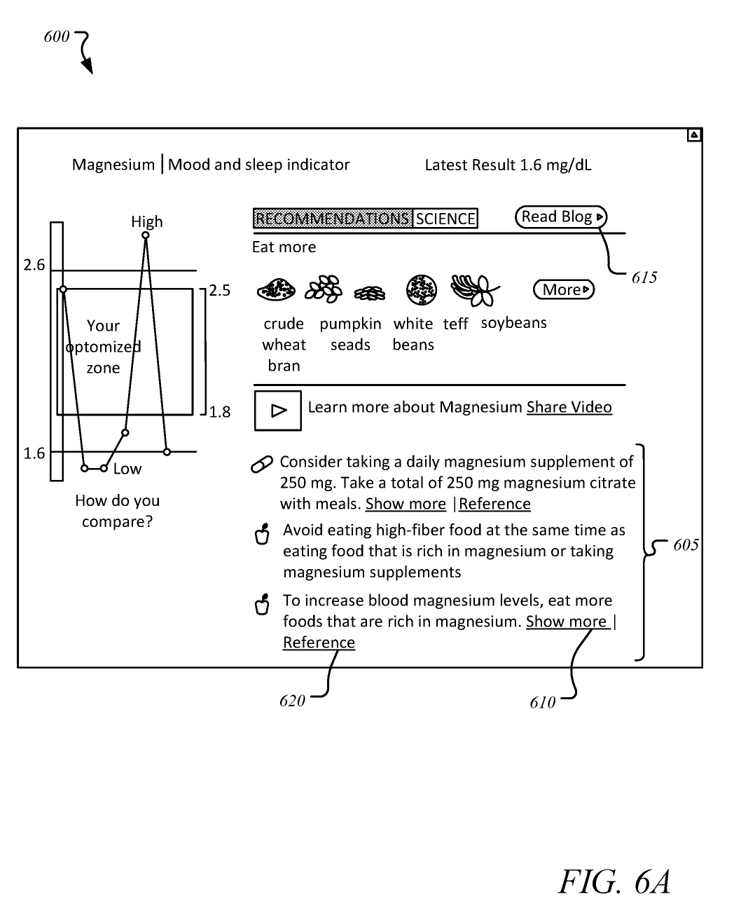

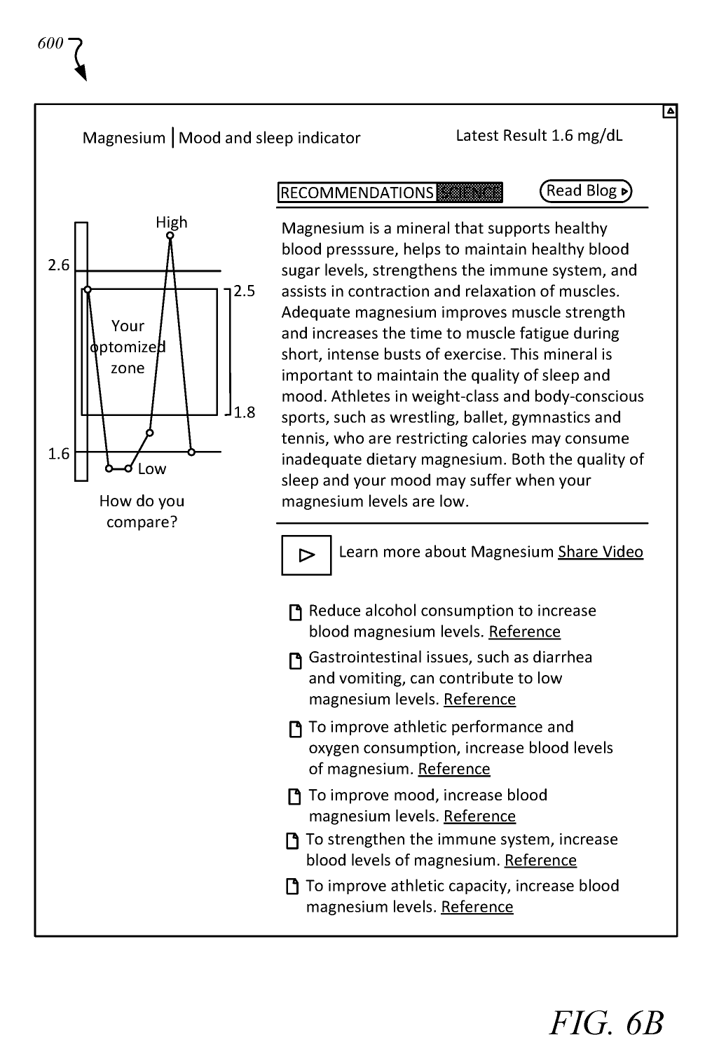

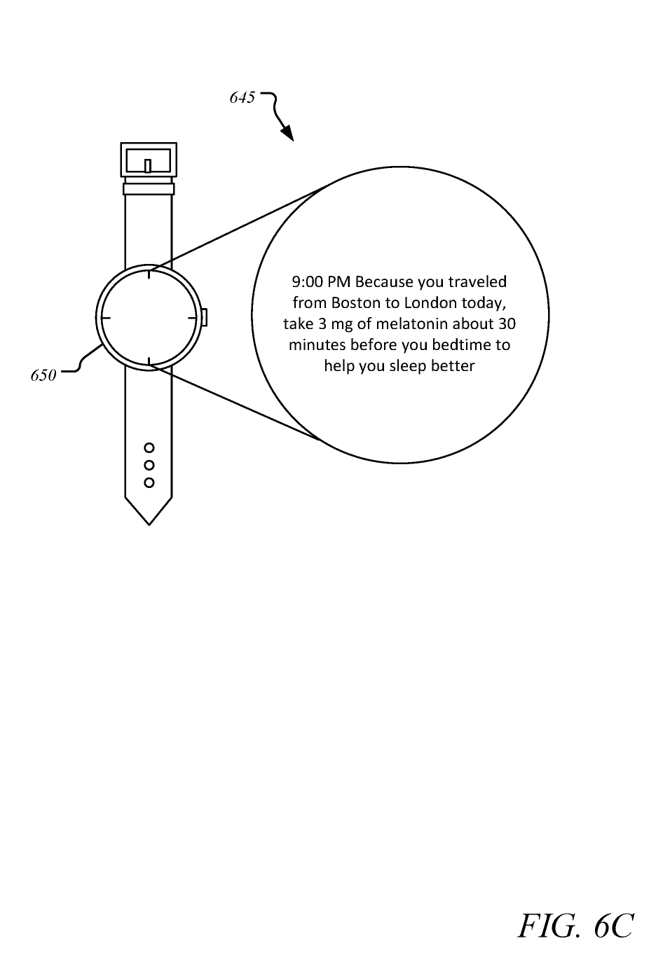

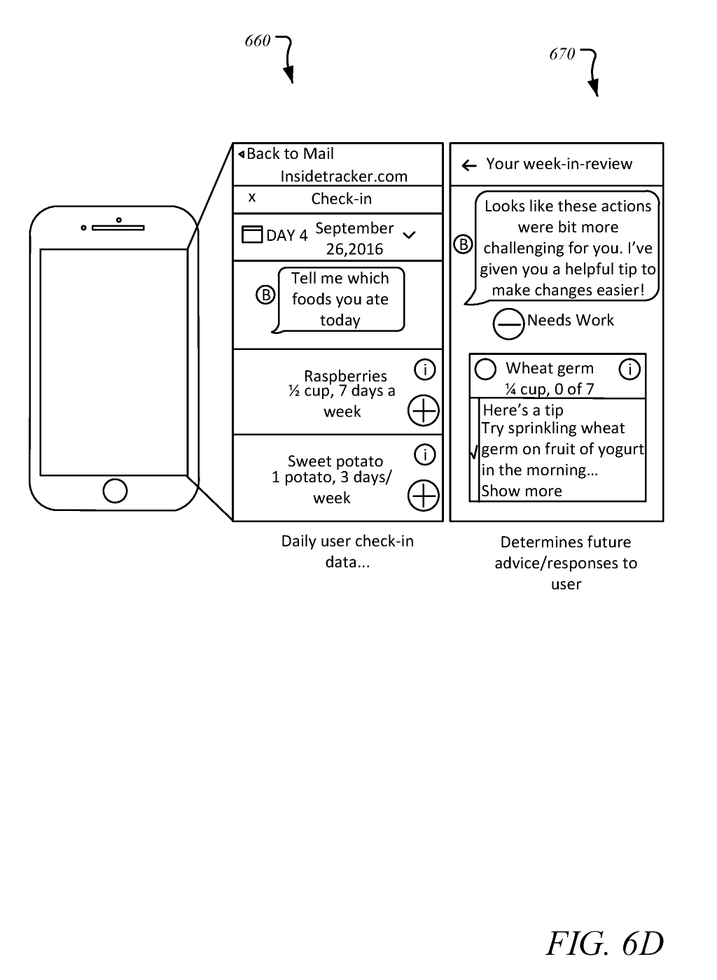

FIGS. 6A-6C are examples of user interfaces for presenting recommendations and information to an end user. In some implementations, the interfaces depicted in FIGS. 6A-6C can be used to provide the personalized recommendations 140 shown in FIG. 1. FIG. 6A shows an interface 600 for providing personalized outputs generated by the analysis engine based on one or more rules from the rules database 105. In some implementations, the interface 600 can include one or more actionable recommendations 605. The interface 600 can also include one or more links to additional information, for example, a link 610 for expanding the recommendations, a link 615 to a web resource (e.g., an explanatory blog post), and/or a link 620 to a source of the one or more rules. The interface 600 can also include one or more images, videos, recipes and tips for implementing the recommendations. The interface 600 can also include one or more calculations to increase the personalization of the rule. For example, there may be a recommendation for a user who is trying to lose weight to consume more protein. The calculation will calculate the daily protein requirement based on the user's weight and activity level. The interface 600 shown in the particular example of FIG. 6B provides educational information such as a paragraph explaining the importance of the biomarker magnesium, the potential issues of having a low level of magnesium, possible symptoms of a low level of magnesium, along with educational statements about magnesium. Other information, as deemed appropriate by back-end personnel such as scientists or system administrators, can be presented using the interface 600. As such, the back-end personnel may create and store various output files together with attributes (e.g., to specify the nature and location of the content of the files in the interface 600), and the analysis engine can be configured to retrieve the appropriate output files based on the rules.

In some implementations, an interface for presenting recommendations and information to a user may be provided on a mobile device such as a smartphone, tablet, e-reader, fitness tracking device, or watch. FIG. 6C depicts an example when an interface 645 is presented on a watch 650. In some implementations, the mobile device can be configured to transmit location information to the analysis engine 125. In such cases, the analysis engine can be configured to provide personalized recommendations based also on the location information. For example, if a user travels from one continent to another, the analysis engine can be configured to infer, based at least in part on received location information, that the user may be suffering from jetlag, and send information on mitigating jetlag for presentation on the mobile device. In some implementations, the end user may see notifications from the analysis engine based on one or more of: time information, location of the user, biomarker data, physiological data, and health- and lifestyle parameters. One or more constraints for presenting the display notifications may be provided to the analysis engine 125, for example, at the time a corresponding rule is created or edited. FIG. 6D illustrates user-interfaces 660 and 670 that may be presented on a mobile device of user. The user-interfaces 660 and 670 may be presented by executing an application (e.g., an “app” that is downloadable from an online repository) on the mobile device of the user.

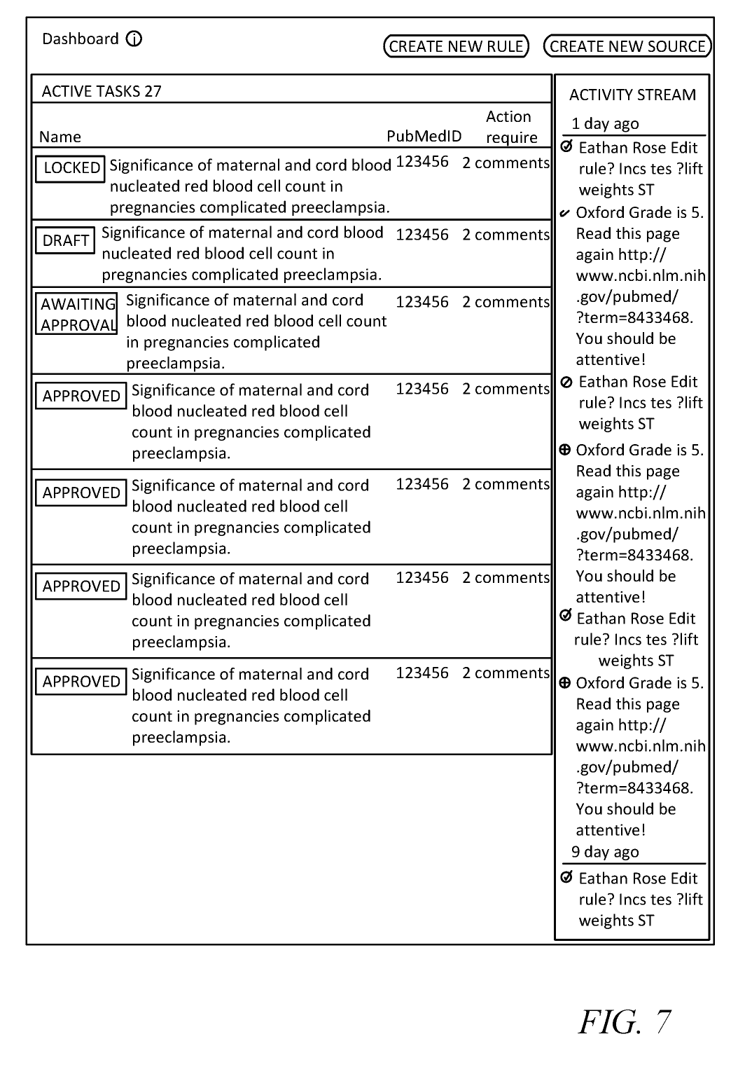

FIG. 7 is an example of a user interface 700 for presenting a dashboard associated with the rules. In some implementations, the interface 700 includes a list of active tasks. The list can indicate, for example, if any user (e.g., a reviewer or administrator) has provided comments on a particular rule. For example, if a reviewer or administrator has commented on a particular rule, the interface 700 can be configured to display such information, thereby indicating to an author of the rule that follow up is needed. In some implementations, the interface 700 can include a representation of an activity stream showing a timeline of activities associated with one or more rules.

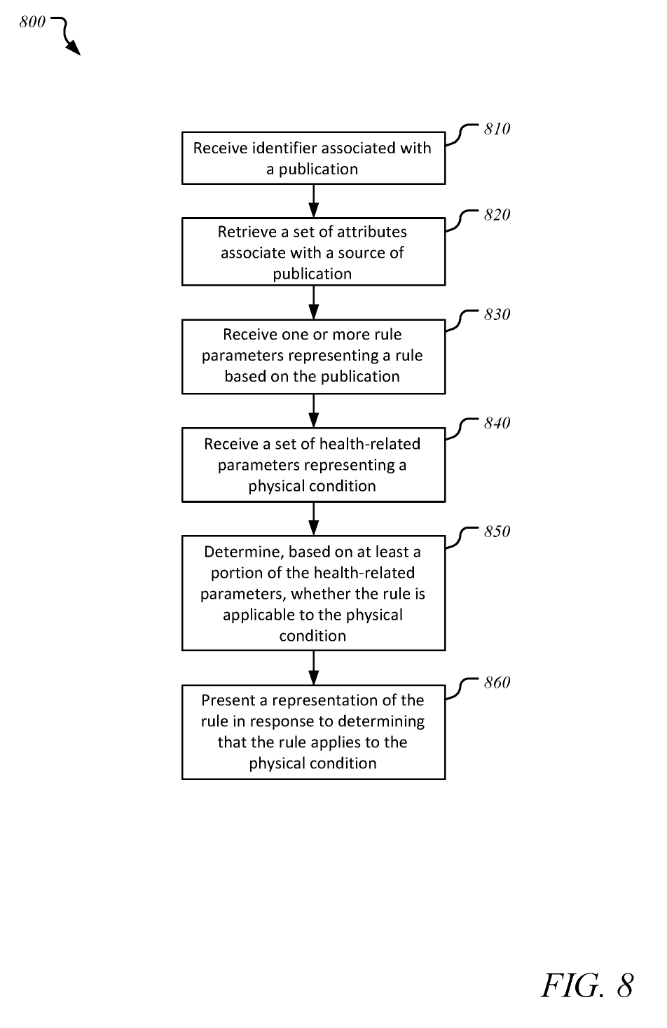

FIG. 8 is a flowchart representing an example process for managing the rules. In some implementations, at least a portion of the process is executed at the analysis engine 125 described above with reference to FIG. 1. The operations of the process 800 includes receiving an identifier associated with a publication (810). The identifier can be received via a user interface presented on a computing device. Examples of such an interface can include the interface 200 described above with reference to FIG. 2A. In some implementations, the identifier includes a standard identifier (e.g., a PubMed identifier) associated with a particular organization. In some implementations, the identifier includes a hyperlink to storage location where a copy of the publication is stored. The identifier can also include a filename associated with an attachment that includes the publication.

Operations of the process 800 also includes retrieving, based on the identifier, a set of attributes associated with the publication (820). The attributes can include, for example, the attributes 205 described with reference to FIG. 2A. For example, the attributes can include one or more of: a title of the publication, an identification of a journal associated with the publication, a date associated with the publication, an author of the publication, an institution associated with the publication, an impact factor of a journal associated with the publication, and a number of citations associated with the publication. Retrieving the set of attributes can include, for example, accessing an electronic repository (e.g., the publication database 130 described with reference to FIG. 1) storing information about the publication, and retrieving at least a portion of the set of attributes from the electronic repository responsive to identifying the publication using the identifier. In some implementations, retrieving the attributes can also include accessing an electronic copy of the publication, and retrieving at least a portion of the set of attributes from the electronic copy of the publication using an optical character recognition (OCR) process.

The process 800 further includes receiving one or more rule parameters representing a rule based on the publication (830). The one or more rule parameters can be received, for example, via a user interface such as the user interface 300 described above with reference to FIGS. 3A and 3B. The one or more rule parameters can include, for example, at least one of: a set of one or more biomarkers associated with the rule, a set of one or more physiological markers associated with the rule, and a set of one or more intermediate parameters associated with the rule. In some implementations, the one or more rule parameters can include at least one condition defined based on the set of one or more biomarkers, the set of one or more physiological markers, and/or the set of one or more intermediate parameters. The condition can be used to determine whether the rule is applicable to the physical condition. The one or more rule parameters can also include information on an output associated with the rule. This can include, for example, the various types of outputs described above with reference to FIGS. 6A and 6B. In some implementations, the one or more rule parameters can be adjusted, for example by presenting a control (e.g., via a user interface) for initiating edits to the one or more rule parameters.

Operations of the process 800 also includes receiving a set of health-related parameters representing a physical condition (840), and determining, based on at least a portion of the health-related parameters and the one or more rule parameters, whether the rule is applicable to the physical condition (850). In some implementations, one or more additional rules can be retrieved from a rules database, and the one or more of the health-related parameters can be used to determine that the one or more additional rules are also applicable to the physical condition. In some implementations, the operations may also include receiving information on one or more user characteristics for a user associated with the physical condition. A determination can then be made, based at least on a portion of the information on the one or more user characteristics, whether the rule is applicable for the user. The information on the one or more user characteristics can include demographic information and/or information on user behavior. In some implementations, a recommendation for improving the physical condition can also be generated based on one or more rules, and possibly also on the information on the one or more user characteristics. In some implementations, the set of health-related parameters can be received via a user interface displayed on a computing device. In some implementations, the set of health parameters can be based on measurements obtained using a mobile device such a smartwatch or smartphone.

The operations further include displaying the rule as a part of a list of rules in response to determining that the rule applies to the physical condition (860). In some implementations, the list of rules can include one or more additional rules also applicable to the physical condition. In some implementations, a rank of the rule within the list can also be determined. The rank determination can be, for example, based on at least a portion of the set of attributes associated with the publication. In some implementations, the rank determination can be, for example, based on a degree of applicability of the rule to the physical condition. In some implementations, the list of rules can be displayed via an interface such as the interface 500 described above with reference to FIG. 5.

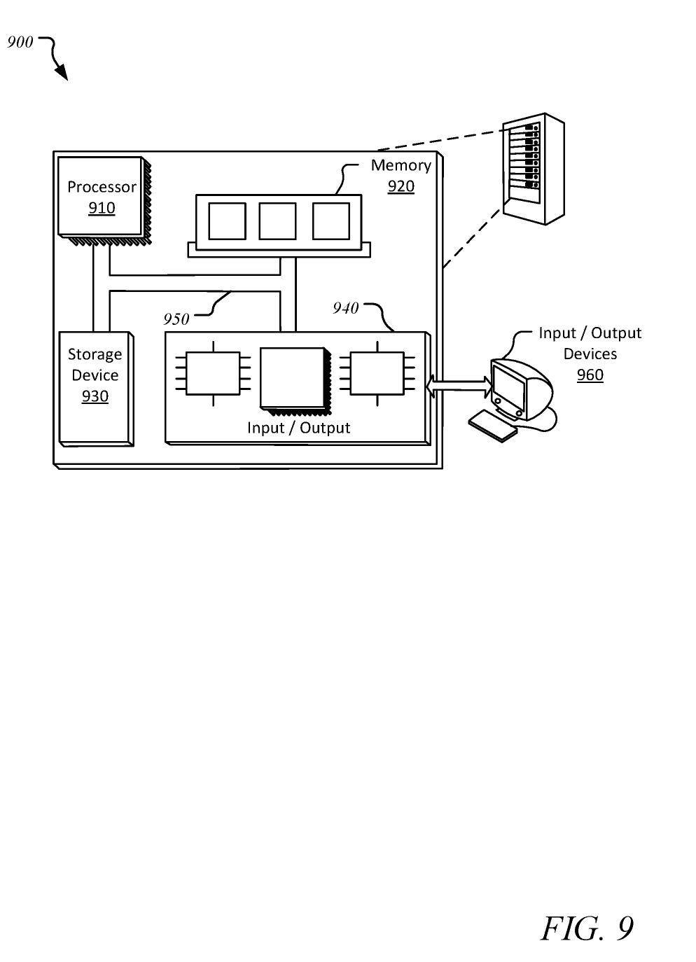

FIG. 9 is block diagram of an example computer system 900 that may be used in performing the processes described herein. For example, the analysis engine 125 described above with reference to FIG. 1, can include at least portions of the computing device 900 described below. Computing device 900 is intended to represent various forms of digital computers, such as laptops, desktops, workstations, servers, blade servers, mainframes, and other appropriate computers. Computing device 900 is further intended to represent various typically non-mobile devices, such as televisions or other electronic devices with one or more processers embedded therein or attached thereto. Computing device 900 also represents mobile devices, such as personal digital assistants, touchscreen tablet devices, e-readers, cellular telephones, smartphones, smartwatches, and fitness tracking devices.

The system 900 includes a processor 910, a memory 920, a storage device 930, and an input/output module 940. Each of the components 910, 920, 930, and 940 can be interconnected, for example, using a system bus 950. The processor 910 is capable of processing instructions for execution within the system 900. In one implementation, the processor 910 is a single-threaded processor. In another implementation, the processor 910 is a multi-threaded processor. The processor 910 is capable of processing instructions stored in the memory 920 or on the storage device 930.

The memory 920 stores information within the system 900. In one implementation, the memory 920 is a computer-readable medium. In one implementation, the memory 920 is a volatile memory unit. In another implementation, the memory 920 is a non-volatile memory unit.

The storage device 930 is capable of providing mass storage for the system 900. In one implementation, the storage device 930 is a computer-readable medium. In various different implementations, the storage device 930 can include, for example, a hard disk device, an optical disk device, or some other large capacity storage device.

The input/output module 940 provides input/output operations for the system 900. In one implementation, the input/output module 940 can include one or more of a network interface device, e.g., an Ethernet card, a serial communication device, e.g., an RS-232 port, and/or a wireless interface device, e.g., and 802.11 card. In another implementation, the input/output device can include driver devices configured to receive input data and send output data to other input/output devices, e.g., keyboard, printer and display devices 960.

The web server, advertisement server, and impression allocation module can be realized by instructions that upon execution cause one or more processing devices to carry out the processes and functions described above. Such instructions can comprise, for example, interpreted instructions, such as script instructions, e.g., JavaScript or ECMAScript instructions, or executable code, or other instructions stored in a computer readable medium. The web server and advertisement server can be distributively implemented over a network, such as a server farm, or can be implemented in a single computer device.

Example computer system 900 can include a server. Various servers, which may act in concert to perform the processes described herein, may be at different geographic locations, as shown in the figure. The processes described herein may be implemented on such a server or on multiple such servers. As shown, the servers may be provided at a single location or located at various places throughout the globe. The servers may coordinate their operation in order to provide the capabilities to implement the processes.

Although an example processing system has been described in FIG. 9, implementations of the subject matter and the functional operations described in this specification can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, e.g., one or more modules of computer program instructions encoded on a tangible program carrier, for example a non-transitory computer-readable medium, for execution by, or to control the operation of, a processing system. The non-transitory computer readable medium can be a machine readable storage device, a machine readable storage substrate, a memory device, or a combination of one or more of them.

In this regard, various implementations of the systems and techniques described herein can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or a combination of such back end, middleware, or front end components. The components of the system can be interconnected by a form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Content, such as ads and GUIs, generated according to the processes described herein may be displayed on a computer peripheral (e.g., a monitor) associated with a computer. The display physically transforms the computer peripheral. For example, if the computer peripheral is an LCD display, the orientations of liquid crystals are changed by the application of biasing voltages in a physical transformation that is visually apparent to the user. As another example, if the computer peripheral is a cathode ray tube (CRT), the state of a fluorescent screen is changed by the impact of electrons in a physical transformation that is also visually apparent. Moreover, the display of content on a computer peripheral is tied to a particular machine, namely, the computer peripheral.

For situations in which the systems and methods discussed here collect personal information about users, or may make use of personal information, the users may be provided with an opportunity to control whether programs or features that may collect personal information (e.g., information about a user's calendar, social network, social actions or activities, a user's preferences, or a user's current location), or to control whether and/or how to receive content that may be more relevant to (or likely to be clicked on by) the user. In addition, certain data may be anonymized in one or more ways before it is stored or used, so that personally identifiable information is removed when generating monetizable parameters (e.g., monetizable demographic parameters). For example, a user's identity may be anonymized so that no personally identifiable information can be determined for the user, or a user's geographic location may be generalized where location information is obtained (such as to a city, ZIP code, or state level), so that a particular location of a user cannot be determined. Thus, the user may have control over how information is collected (and/or used) about him or her.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, various modifications may be made without departing from the spirit and scope of the disclosure. For example, various forms of the flows shown above may be used, with steps re-ordered, added, or removed. Accordingly, other implementations may fall within the scope of the following claims.

### APPENDIX A: CORE SNPS

RSID
rs10830963
rs12255372
rs1260326
rs13266634
rs1360780
rs1421085
rs1572312
rs16827466
rs17300539
rs17342717
rs17782313
rs1780324
rs1799752
rs1799865
rs1800562
rs1800849
rs1800961
rs1801133
rs1801282
rs1815739
rs2016520
rs2070744
rs2132570
rs2138852
rs2187668
rs2281135
rs2282679
rs2497306
rs3093059
rs3196378
rs324981
rs328
rs361525
rs3808607
rs3811647
rs3917878
rs3918358
rs4149056
rs429358
rs4340
rs4420638
rs4646994
rs4680
rs4713916
rs4895441
rs4988235
rs5082
rs540467
rs560887
rs601338
rs602662
rs6195
rs6196
rs6258
rs657152
rs660339
rs662799
rs7181230
rs727428
rs7385804
rs7553007
rs768539
rs769449
rs780093
rs780094
rs7903146
rs7924316
rs8078723

### APPENDIX B: LIST OF USABLE SNPS

RSID
rs10163409
rs10168349
rs10224002
rs1042031
rs10454142
rs10455872
rs10482605
rs10491334
rs10494326
rs10495024
rs10495928
rs10495960
rs1050828
rs10521222
rs10737277
rs10741657
rs10745954
rs1075023
rs10752979
rs10757278
rs10758658
rs10761779
rs10778213
rs10811679
rs10818854
rs10822184
rs10830963
rs10882272
rs10903129
rs10937273
rs10986018
rs11023374
rs11065987
rs11083750
rs11085824
rs11132733
rs1122794
rs11239550
rs113459440
rs11549465
rs11591741
rs11597086
rs11597390
rs11603334
rs11605924
rs11645428
rs1169313
rs11761528
rs117672662
rs11829037
rs1183910
rs11915082
rs12037222
rs1205
rs12059860
rs12075
rs12086634
rs12150660
rs121918392
rs121918393
rs121918394
rs121918395
rs121918396
rs121918397
rs121918398
rs121918399
rs12239046
rs12239267
rs12255372
rs12355784
rs12405556
rs12485738
rs12594956
rs1260326
rs12649507
rs12661667
rs12718597
rs12722
rs12982192
rs131794
rs13233571
rs13266634
rs13335629
rs1341666
rs13447445
rs1360780
rs1378942
rs140808909
rs1408272
rs1421085
rs1532624
rs1569480
rs1572312
rs1573036
rs1667255
rs16842520
rs16842525
rs16881983
rs16926246
rs1695
rs17064262
rs17141006
rs17231506
rs172629
rs17277546
rs17300539
rs17342717
rs174537
rs174547
rs174548
rs174549
rs174550
rs174556
rs174561
rs174577
rs174583
rs17496332
rs1761667
rs17696736
rs17782313
rs1780324
rs1799752
rs1799865
rs1799884
rs1799945
rs1799971
rs1799983
rs1800206
rs1800445
rs1800546
rs1800562
rs1800592
rs1800775
rs1800795
rs1800796
rs1800797
rs1800849
rs1800961
rs1801133
rs1801282
rs1805018
rs1815739
rs182549
rs1870377
rs1887582
rs1892534
rs190853081
rs1937
rs197273
rs199768005
rs1999594
rs2000999
rs2010963
rs2016520
rs201672011
rs2070744
rs2075650
rs2075671
rs2097677
rs2132570
rs2138852
rs2143571
rs2185570
rs2187668
rs2213169
rs2228145
rs2228467
rs2229309
rs2247215
rs2252673
rs225918
rs2259816
rs2273238
rs2281135
rs2282679
rs2301475
rs2393791
rs2395182
rs2411984
rs2412522
rs2413450
rs2414096
rs2470152
rs2470158
rs2470893
rs2472297
rs2472300
rs2479106
rs2483058
rs2494250
rs2497306
rs2540917
rs2637125
rs267606661
rs267606662
rs267606663
rs267606664
rs2698530
rs2712381
rs2794520
rs2808629
rs2836878
rs285
rs28931576
rs28931577
rs28931578
rs28931579
rs293428
rs2979481
rs3091244
rs3093058
rs3093059
rs3093077
rs3135506
rs3196378
rs324640
rs324981
rs328
rs340029
rs342293
rs356653
rs361525
rs3748085
rs3779195
rs3798220
rs3800737
rs3808607
rs3811647
rs3820185
rs3834458
rs3845624
rs387906567
rs387906568
rs3890182
rs3917878
rs3918358
rs3923809
rs397514253
rs397514254
rs405509
rs4128725
rs4129267
rs4133289
rs41380347
rs4149056
rs41525747
rs4244372
rs424971
rs4253778
rs429358
rs4340
rs4343
rs439401
rs440446
rs440837
rs4410790
rs4420065
rs4420638
rs4466998
rs449647
rs4588
rs4639334
rs4646903
rs4646994
rs4654748
rs4657616
rs4680
rs4713586
rs4713916
rs4778889
rs4812829
rs4815670
rs4820268
rs4820599
rs4895441
rs4903031
rs4904670
rs4910472
rs4982603
rs4988235
rs4994
rs505151
rs5082
rs5128
rs514708
rs5219
rs527236160
rs5400
rs540467
rs552148
rs560887
rs56149945
rs5751876
rs5756504
rs579459
rs5918
rs592423
rs5934505
rs5987027
rs601338
rs6013509
rs6022662
rs6025
rs602662
rs6166
rs61736690
rs61739327
rs6190
rs6196
rs6235
rs6257
rs6258
rs6265
rs6277
rs628751
rs641959
rs643381
rs6552828
rs6564851
rs6569992
rs660339
rs662799
rs66468343
rs6667499
rs6720173
rs672316
rs6734238
rs6738028
rs6746030
rs680
rs6858749
rs6923761
rs693
rs6968865
rs7023923
rs7041
rs7144481
rs7181230
rs7181866
rs7189020
rs7191721
rs7203560
rs7255045
rs726640
rs727428
rs7310409
rs7385804
rs740160
rs7412
rs7439293
rs7454108
rs7531832
rs7553007
rs760241
rs762551
rs7672337
rs768539
rs769446
rs769449
rs769450
rs769452
rs769455
rs7732671
rs7775228
rs7775698
rs7776054
rs7786877
rs7787204
rs780093
rs780094
rs7894051
rs7903146
rs7910927
rs7924316
rs7961894
rs8023580
rs8031031
rs8078723
rs8176720
rs818571
rs8192678
rs838133
rs838145
rs854561
rs855791
rs857721
rs898611
rs909814
rs9253
rs9289231
rs9344950
rs9349205
rs9349379
rs9357271
rs9362667
rs9373124
rs9374080
rs9375813
rs9376092
rs9461011
rs9467160
rs9468304
rs9483788
rs9494145
rs9628671
rs964184
rs9859260
rs987710
rs9905820
rs9939609
rs9982601
rs9987289
rs2266633

1. A computer-implemented method comprising:
receiving, via an interface displayed on a computing device, an identifier associated with a publication;
retrieving, by one or more processing devices based on the identifier, a set of attributes associated with the publication;
receiving, via the interface, one or more rule parameters representing a rule based on the publication;
receiving a set of health-related parameters representing a physical condition;
determining, by the one or more processing devices based on at least a portion of the health-related parameters and the one or more rule parameters, whether the rule is applicable to the physical condition; and
presenting a representation of the rule on an output device as a part of a list of rules in response to determining that the rule applies to the physical condition.

2. The method of claim 1, wherein the list of rules comprises one or more additional rules also applicable to the physical condition.

3. The method of claim 2, further comprising determining a rank of the rule within the list.

4. The method of claim 3, wherein the rank is determined based on at least a portion of the set of attributes associated with the publication.

5. The method of claim 3, wherein the rank is determined based on a degree of applicability of the rule to the physical condition.

6. The method of claim 1, further comprising displaying, on the interface, a control for initiating edits to the one or more rule parameters.

7. The method of claim 1, wherein the one or more rule parameters comprise at least one of: a set of one or more biomarkers associated with the rule, a set of one or more physiological markers associated with the rule, a set of one or more genetic markers associated with the rule, a set of demographic and behavior data, and a set of one or more intermediate parameters associated with the rule.

8. The method of claim 7, wherein the one or more rule parameters comprise at least one condition defined based on the set of one or more biomarkers, the set of one or more genetic markers, the set of one or more physiological markers, and/or the set of one or more intermediate parameters, wherein the condition is used to determine whether the rule is applicable to the physical condition.

9. The method of claim 1, wherein the one or more rule parameters comprise information on an output associated with the rule.

10. The method of claim 1, further comprising:
retrieving one or more additional rules from a rules database stored on one or more storage devices; and
determining, based on one or more of the health-related parameters, that the one or more additional rules are also applicable to the physical condition.

11. The method of claim 1, further comprising generating, based on the rule, a recommendation for improving the physical condition.

12. The method of claim 1, further comprising storing a representation of the rule in a database.

13. The method of claim 1, wherein the identifier comprises at least one of: i) a PubMed identifier associated with the publication, ii) a hyperlink to a storage location, or iii) a filename associated with an attachment that includes the publication.

14. The method of claim 1, wherein the set of attributes includes one or more of: a title of the publication, an identification of a journal associated with the publication, a date associated with the publication, an author of the publication, and an institution associated with the publication.

15. The method of claim 1, wherein the set of attributes includes an impact factor of a journal associated with the publication.

16. The method of claim 1 further comprising:
receiving information on one or more user characteristics for a user associated with the physical condition; and
determining, based at least on a portion of the information on the one or more user characteristics, whether the rule is applicable for the user.

17. The method of claim 16, wherein the information on one or more user characteristics comprises at least one of: demographic information and information on user behavior.

18. The method of claim 1, wherein retrieving the set of attributes comprises:
accessing an electronic repository storing information about the publication; and
retrieving at least a portion of the set of attributes from the electronic repository responsive to identifying the publication using the identifier.

19. The method of claim 1, wherein retrieving the set of attributes comprises:
accessing an electronic copy of the publication; and
retrieving at least a portion of the set of attributes from the electronic copy of the publication using an optical character recognition (OCR) process.

20. The method of claim 1, wherein the set of health-related parameters is received via a user interface displayed on a computing device.

21. The method of claim 1, wherein the set of health parameters is based on measurements obtained using a wearable device.

22. A system comprising:
a memory device; and
an analysis engine comprising one or more processing devices, the analysis engine configured to:
receive, via an interface displayed on a computing device, an identifier associated with a publication,
retrieve, based on the identifier, a set of attributes associated with the publication,
receive, via the interface, one or more rule parameters representing a rule based on the publication,
receive a set of health-related parameters representing a physical condition,
determine, based on at least a portion of the health-related parameters and the one or more rule parameters, whether the rule is applicable to the physical condition, and
present a representation of the rule on an output device as a part of a list of rules in response to determining that the rule applies to the physical condition.

23.-42. (canceled)

43. One or more machine-readable storage devices storing instructions that are executable by one or more processing devices to perform operations comprising:
receiving, via an interface displayed on a computing device, an identifier associated with a publication;
retrieving, based on the identifier, a set of attributes associated with the publication;
receiving, via the interface, one or more rule parameters representing a rule based on the publication;
receiving a set of health-related parameters representing a physical condition;
determining, based on at least a portion of the health-related parameters and the one or more rule parameters, whether the rule is applicable to the physical condition; and
presenting a representation of the rule on an output device as a part of a list of rules in response to determining that the rule applies to the physical condition.

44.-59. (canceled)
